(19) United States
(12) Patent Application Publication
(10) Pub. No.: US 2025/0372254 A1
(43) Pub. Date: Dec. 4, 2025

(54) CLINICAL SUMMARY SYSTEMS AND METHODS

(71) Applicant: Iodine Software, LLC, Austin, TX (US)

(72) Inventors: Edward Peter Coombes, Plymouth, MA (US); Bryan Joseph Horne, Highlands Ranch, CO (US); Chelsey Rae Wildenborg, Denver, CO (US); Dillon Paul LeDoux, Austin, TX (US); Brandon Vecchio, Austin, TX (US); Kathleen Abbe, Buda, TX (US)

(21) Appl. No.: 19/221,351

(22) Filed: May 28, 2025

Related U.S. Application Data
(60) Provisional application No. 63/652,469, filed on May 28, 2024.

(51) Int. Cl. G16H 50/20; G16H 10/60; G16H 15/00

(52) U.S. Cl. CPC G16H 50/20; G16H 10/60; G16H 15/00

(57) ABSTRACT

For each respective patient of a plurality of prediction-eligible patients of a healthcare provider, a computer determines, based on observations of the plurality of prediction-eligible patients, an admit status prediction (ASP) for the respective patient. Utilizing the ASP and major diagnosis category (MDC) prediction features extracted from patient visits eligible for the ASP, the computer further determines a MDC prediction (MDCP) for the respective patient and extracts, from a database, clinical data for the respective patient. The computer maps, utilizing the MDCP, individual clinical data points in the clinical data for the respective patient thus extracted into grouped items and generates a visit summary for the respective patient. The visit summary includes the ASP, the MDCP, and an explanation of how the ASP and the MDCP were made based at least on the grouped items.

### CROSS-REFERENCE TO RELATED APPLICATION(S)

This application claims a benefit of priority under 35 U.S.C. § 119(e) from Provisional Application No. 63/652,469, filed May 28, 2024, entitled “CLINICAL SUMMARY SYSTEMS AND METHODS,” the entire disclosure of which is fully incorporated by reference herein for all purposes.

### TECHNICAL FIELD

This disclosure generally relates to data analyses and machine learning. More particularly, this disclosure relates to digesting outputs from predictive data analyses and utilizing machine learning to explain the predictive outputs in a concise clinical summary.

### BACKGROUND OF THE RELATED ART

Today, when patients are admitted to hospitals, they will receive a particular admission status. That admission status is based on the patient's severity of illness and intensity of services required. The highest level of care is Inpatient status, with Outpatient or Observation being a lower level of service. These patient statuses reflect different billing needs and requirements, but do not prevent a patient from receiving the care required.

In a hospital, a utilization management (UM) nurse or a utilization review (UR) nurse (which, for the sake of convenience, is also referred to hereinafter as a “UM nurse”) is usually in charge of monitoring patient care and help controlling costs for various utilization of facilities and agencies in the hospital. Throughout a patient's stay in the hospital (which is referred to herein as a “visit”), UM nurses are responsible for communicating clinical updates to the insurance companies (sometimes before, prior-authorization) about the patient's status and progress. Insurance companies use this clinical data to justify continuing to pay for a given-level of care, or, will deem those services not medically necessary and deny the claim. There are a variety of methods for communicating these clinical updates to insurance companies, however, these legacy methods are not easily reviewable, presentable, or curated.

Part of a UM nurse's job is to review each patient and determine the correct status for the patient. If the UM nurse believes that a patient's status should change, the UM nurse needs to build a case for the status change and then presents it to the patient's attending physician. Given that timing is a major component, the UM nurse needs to optimize his/her time between reviewing charts and contacting physicians.

However, in order to correctly determine the status of a patient, the patient has to be clinically reviewed. The scope of a clinical review is vast, including, but is not limited to, documents such as nurse's notes, physician notes, etc. that document the patient's care during a visit, lab results from works done on the patients, tests performed on the patients, the patient's vitals taken at various places and/or time points, and so on. As the patient is being observed, the severity of their symptoms and the severity of their illness play a big part in determining whether their status should be Inpatient or Outpatient.

Unfortunately, the UM nurse has a limited amount of time to fully review each patient. Therefore, often times, a patient's status is determined based on incomplete data and/or subjectively based on the UM's experience.

Without a quantifiable way to determine whether to move a patient from Inpatient to Outpatient, or vice versa, on the one hand, the patient may not receive the right care (e.g., when the patient should be Inpatient, but is kept as Outpatient or Observation) and, on the other hand, it may be a waste of resources (e.g., when the patient should be Outpatient or Observation, but is moved to Inpatient). Often times, no one can concisely explain why a patient is moved from Inpatient to Outpatient, or vice versa.

In view of the foregoing, there is a need for innovations and improvements in providing a clinical summary that can concisely explain a patient's status, including the patient's predicted admit status. The invention disclosed herein can address this need and more.

### SUMMARY OF THE DISCLOSURE

The invention disclosed herein can facilitate a more efficient use of resources and streamline workflows involving reviews of patient records, essentially eliminating the need to review patient cases that have a low chance of status change and that are clinically in the most appropriate status. In turn, the invention can allow UM nurses to use their time more efficiently. With the invention disclosed herein, a patient's status can be quantifiably, reproducibly, reliably determined and concisely explained based on data without having to rely on a user's input (e.g., an UM nurse's experience).

A system implementing the invention disclosed here automatically collects and presents key clinical data points that UM nurses can use to communicate with interested parties (e.g., insurance providers). The disclosed patient status prediction method can save review time by determining and presenting patients who have a high probability of an incorrect status, thereby allowing UM nurses to focus on those patients and, in turn, be able to work more efficiently. In some cases, the UM nurses can edit and/or provide additional information before officially submitting their findings in a clinical summary to the interested parties.

A goal of this disclosure is to provide a data-driven, concise way to explain a patient's predicted status, focusing specifically on the transitions between inpatient and observation statuses. The invention recognizes the distinct reasons that facilitate a patient's transition from Observation (or Outpatient) to Inpatient, and vice versa.

To achieve this goal, the invention disclosed herein leverages machine learning models to learn the nuanced characteristics of patients that are in an Observation status or Inpatient status. Recognizing the reasons for an Observation patient transitioning to Inpatient are typically different from an Inpatient patient moving to Observation, the invention disclosed herein particularly utilizes two distinct binary classification models to capture these differences more effectively.

Each binary classification model takes a patient's current status and predicts the likelihood for a change in status. For example, if a patient is in Observation, a first model predicts the likelihood that the patient should either be admitted as Inpatient or discharged from the hospital. If a patient is an Inpatient, a second model predicts whether the patient should be moved to Observation or be discharged. Each model outputs a patient's status prediction. If the predicted status indicates that the patient is not (but should be) in the hospital, the predicted patient status can be reported out (e.g., to an administrator of the hospital through appropriate communication channels) and/or prioritized for review (e.g., through a user interface or dashboard of a system implementing an embodiment disclosed herein). Such a more accurate patient status prediction can effectively reduce the impact on the patient as well as hospital resources due to incorrect patient status.

The patient's admission status prediction and data extracted from the patient's records can be used to predict the patient's medical diagnosis category. The patient's admission status prediction and the patient's medical diagnosis category can be used to generate a visit summary for the patient, along with explanations that concisely describe the reasons for the patient's predicted medical diagnosis category. The visit summary, in turn, can be used to generate a document, referred to herein as a clinical summary, that can be distributed or otherwise used across a network.

In some embodiments, a method disclosed herein may perform, for each respective patient of a plurality of prediction-eligible patients of a healthcare provider, determining, by a first machine learning model based on observations of the plurality of prediction-eligible patients, an admit status prediction (ASP) for the respective patient, determining, by a second machine learning model utilizing the ASP and major diagnosis category (MDC) prediction features extracted from patient visits eligible for the ASP, a MDC prediction (MDCP) for the respective patient; and extracting, from a database, clinical data for the respective patient. In some embodiments, the first machine learning model comprises a multi-class classifier and the second machine learning model comprises a binary classifier. In some embodiments, the observations can include at least one of: a date when a current status was established, a date when the current status was changed, any change in severity of a patient's illness, or any change in the severity of the patient's symptoms.

In some embodiments, the method may further comprise mapping, utilizing the MDCP, individual clinical data points in the clinical data for the respective patient thus extracted into grouped items and generating a visit summary for the respective patient. In some cases, the visit summary may include singleton item(s) as well.

As an example, the visit summary can include the ASP, the MDCP, and an explanation of how the ASP and the MDCP were made based at least on the grouped items. In some embodiments, the explanation can comprise a plurality of explanation factors describing the MDC. In some embodiments, the method may include providing a user interface with one or more user interface elements for editing the visit summary. Utilizing the visit summary thus generated and/or user-edited, a clinical summary can then be generated in a distributable document format for communicating to a computing facility (e.g., a client system such as a payer system) or a third-party service provider computer (e.g., a fax server owned or operated by a third party).

One embodiment comprises a system comprising a processor and a non-transitory computer-readable storage medium that stores computer instructions translatable by the processor to perform a method substantially as described herein. Another embodiment comprises a computer program product having a non-transitory computer-readable storage medium that stores computer instructions translatable by a processor to perform a method substantially as described herein. Numerous other embodiments are also possible.

These, and other, aspects of the disclosure will be better appreciated and understood when considered in conjunction with the following description and the accompanying drawings. It should be understood, however, that the following description, while indicating various embodiments of the disclosure and numerous specific details thereof, is given by way of illustration and not of limitation. Many substitutions, modifications, additions, and/or rearrangements may be made within the scope of the disclosure without departing from the spirit thereof, and the disclosure includes all such substitutions, modifications, additions, and/or rearrangements.

### BRIEF DESCRIPTION OF THE DRAWINGS

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIG. 1 is a diagram that illustrates a system with a prediction cycle controller according to some embodiments disclosed herein.

FIG. 2 is a flow chart that illustrates an example of operation performed by the prediction cycle controller according to some embodiments disclosed herein.

FIG. 3 is a process diagram that illustrates an example of the prediction cycle controller in operation according to some embodiments disclosed herein.

FIG. 4 is a flow diagram that illustrates an example of a process for training and using machine learning models to generate an admit status prediction according to some embodiments disclosed herein.

FIG. 5 is an example of what summary items may be grouped together and what summary items may be presented as singleton items according to some embodiments disclosed herein.

FIG. 6 illustrates an example of a system for generating a clinical summary according to some embodiments disclosed herein.

FIG. 7 depicts a flow diagram that illustrates an example operation for generating a clinical summary according to some embodiments disclosed herein.

FIG. 8 provides an example of a data model used by a clinical summary resolver in operation according to some embodiments disclosed herein.

FIGS. 9A-B show example views of a user interface displaying a machine-generated user-editable clinical summary according to some embodiments disclosed herein.

FIG. 10 depicts a diagrammatic representation of a data processing system for implementing an embodiment disclosed herein.

### DETAILED DESCRIPTION

The invention and the various features and advantageous details thereof are explained more fully with reference to the non-limiting embodiments that are illustrated in the accompanying drawings and detailed in the following description. Descriptions of well-known starting materials, processing techniques, components and equipment are omitted so as not to unnecessarily obscure the invention in detail. It should be understood, however, that the detailed description and the specific examples, while indicating some embodiments of the invention, are given by way of illustration only and not by way of limitation. Various substitutions, modifications, additions, and/or rearrangements within the spirit and/or scope of the underlying inventive concept will become apparent to those skilled in the art from this disclosure.

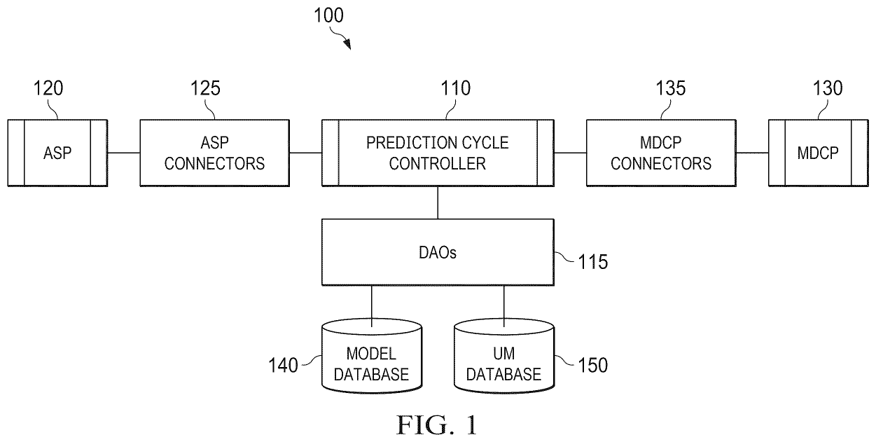

As alluded to above, a patient's admission status prediction and data extracted from the patient's records can be used to predict the patient's medical diagnosis category. Then, the patient's admission status prediction and the patient's medical diagnosis category can be used to generate a visit summary for the patient, along with explanations that concisely describe the reasons for the patient's predicted medical diagnosis category. Based on the visit summary, a clinical summary can be generated and distributed. An example architecture that supports the generation of a clinical summary will now be described with reference to FIGS. 1-4.

FIG. 1 is a diagram that illustrates a system 100 with a prediction cycle controller 110. As a non-limiting example, the system 100 may be implemented using Kubernetes (K8s) in a private cloud. Some or all of the components of the system 100 may be implemented as microservices.

In the example of FIG. 1, the prediction cycle controller 110 uses data access objects (DAOs) 115 to communicate with a model database 140 and a UM database 150. A DAO provides an abstract interface to the model database 140 or to the UM database 150. DAOs are known to those skilled in the art and thus are not further described herein.

Both the model database 140 and the UM database 150 may store patient records, with the majority of the patient records stored in the model database 140 and the UM database stores those that can be accessed by UM nurses. U.S. Pat. No. 10,886,013, entitled “SYSTEMS AND METHODS FOR DETECTING DOCUMENTATION DROP-OFFS IN CLINICAL,” which is incorporated by reference herein, provides examples of the various types of information that can be documented in a patient's record, for instance, texts from the patient's medical documents, lab results, tests given, medications ordered, and cultures information. In some embodiments, the patient's record can include outputs of condition models described in U.S. Pat. No. 10,733,566, entitled “HIGH FIDELITY CLINICAL DOCUMENTATION IMPROVEMENT (CDI) SMART SCORING SYSTEMS AND METHODS,” which is also incorporated by reference herein.

There can be multiple types of condition models for each particular medical condition (e.g., acute renal failure, anemia, chronic renal failure, heart failure, hyponatremia, hypernatremia, myocardial infarction, pancreatitis, pneumonia, respiratory failure, sepsis, shock, stroke, etc.). Each of these medical conditions has an ensemble of condition models. In embodiments, an ensemble may contain a number of condition models, with each condition model comprising a set of factors or indicators that are predictive of whether a particular medical condition is reflected in a patient's record. Outputs from these condition models can be used to evaluate a patient case and generate a prediction, as well as efficiently recognizing patterns within the data so as to optimize the prediction.

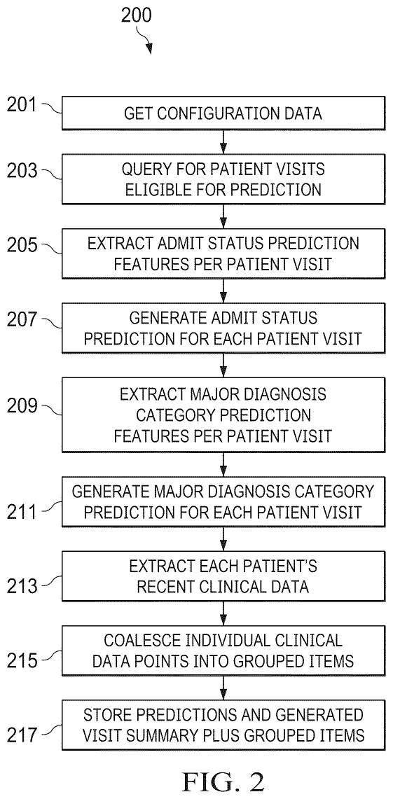

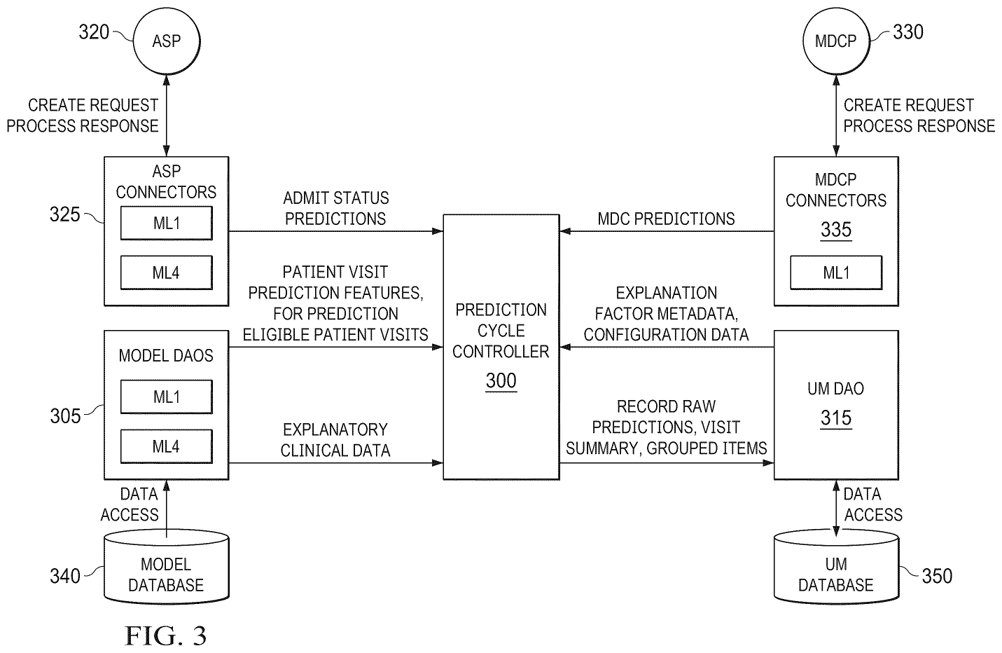

As illustrated in FIG. 1, the prediction cycle controller 110 is communicatively connected to an admit status predictor (ASP) 120 through ASP connector(s) 125 to a major diagnosis category predictor (MDCP) 130 through MDCP connector(s) 135. These are further described below with reference to FIGS. 2-3.

As those skilled in the art can appreciate, Major Diagnostic Categories (MDC) are formed by dividing all possible principal diagnoses into 25 mutually exclusive diagnosis areas, with each MDC corresponding to a major organ system (e.g., Respiratory System, Circulatory System, Digestive System, etc.) rather than a specific disease (e.g., high blood pressure, cancer, etc.). In this disclosure, each respective MDC is associated with a list of explanation factors that describe the respective MDC.

In some embodiments, there can be multiple UM databases, each for an enterprise customer (client). Data from individual client databases (e.g., a volume of electronic medical records (EMRs)) can be pre-processed to pull patient records (data sets) of interest. For instance, the system may use a search key (e.g., vsc=visit_state_change) to find when patients entered and left certain statuses (e.g., searching for Obs for Observations and IP for Inpatients and ignoring others), specifically when patients moved to different statuses (e.g., from Obs to IP or from IP to Obs).

As another example, the system may use the date when a patient was in a certain status (e.g., Obs) to pull data for the patient on that particular date or the system may use what status a patient went to next (e.g., IP) as a training target. Based on the entry date and exit date of a certain status (e.g., Obs or IP), the system can then pull multiple different types of data for machine learning training, e.g., by examining queries to describe the status change from Obs to IP or from IP to Obs.

FIG. 2 is a flow chart that illustrates an example of a prediction cycle 200 performed by the prediction controller described above. In this example, the prediction cycle starts with getting configuration data from the UM database using a UM DAO (201). The configuration data configures how a job is run and can include model versions, confidence thresholds, explanation factor metadata, etc. For example, a job scheduler can be configured to pull patient records (e.g., tens of thousands of patient records per a EMR volume) per customer (e.g., hospital, healthcare provider, etc.) every hour.

Next, the prediction cycle controller queries the model database (e.g., using a model DAO) for patient visits that are eligible for patient admit status prediction (203). This can include checking whether new data has arrived, whether the “cooling off” period has elapsed, whether the Inpatient or Outpatient status has changed, etc. The prediction cycle controller then extracts admit status prediction (ASP) features for each eligible patient visit (205). Examples of ASP features can include various observations such as the date when the current status was established, the date when the status was changed, any change in severity of the patient's illness, any change in the severity of the patient's symptoms, etc.

The prediction cycle controller sends (e.g., through an ASP connector) the extracted data (e.g., more than 40 observations) to the ASP which, in turn, is operable to examine the plurality of observations of prediction-eligible patients of the customer and generate an admit status prediction for each of those patients (207). Each batch may take only a few minutes to process.

The prediction cycle controller also extracts MDCP features from the records of prediction-eligible patients (209). The MDCP features thus extracted may vary from model to model, based on model configurations, as different major diagnosis categories usually have different data points of interest that may contribute to the determination of a MDC. The prediction cycle controller sends (e.g., through a MDCP connector) the extracted data along with the admit status prediction generated by the ASP to the MDCP. The MDCP, in turn, is operable to examine the extracted MDCP features and the admit status prediction generated by the ASP and generate a MDC prediction (211).

Next, the prediction cycle controller extracts the patient's clinical data from the model database (e.g., using the model DAO) (213). Since each MDC is associated with a list of explanation factors, the MDC prediction can be used to map or otherwise coalesce individual clinical data points into grouped items (215). At this point, a visit summary for the patient can be generated. This visit summary, which includes a prediction on a patient's status, a prediction of the patient's major diagnosis category, and explanations of how the predictions were made, can be presented and/or stored (e.g., in the UM database) for use by the UM nurse to make a case for changing the patient's status (217).

FIG. 3 is a process diagram that illustrates an example of a prediction cycle controller 300 in operation. In this example, a prediction cycle begins with getting configuration data. This can be done by using an UM DAO 315 to pull data from an UM database 350.

Then, the prediction cycle controller 300 queries a model database 340 using a model DAO 305. As discussed above, the model database (DB) 340 can store patient records (for one or more clients) and the UM database 350 can store metadata and configuration data that can be used by the prediction cycle controller in controller how each prediction cycle is run. Since the model DB 340 may store patient records for multiple clients (e.g., a hospital, a healthcare provider, etc.), the prediction cycle controller 300 may use a model DAO 305 per a client (e.g., a client-specific model DAO) to pull patient records of the client.

The prediction cycle controller 300 sends the extracted data to an ASP 320 which, based on the extracted data, makes a prediction using a first machine learning model (ML1) or a second machine learning model (ML2), whichever is configured for a particular client. The ASP 320 outputs a multi-class classification (from ML1) or a binary answer (from ML4). Examples of machine learning models are further described below.

The prediction cycle controller 300 then extracts the MDCP features from the model DB 340 and calls (via an MDCP connector 335) using the admit status prediction as input to an MDCP 330. In some embodiments, the prediction cycle controller 300 maintains a list of ASP features and a list of MDCP features, so it knows what to extract so as to provide inputs to the ASP 320 and the MDCP 330.

The prediction cycle controller 300 uses the model DAO(s) 305 to capture data and provides the data thus captured to the ASP 320 to get a prediction and also to the MDCP 330 with the output from the ASP 320 to get a MDC prediction. Once the MDCP 330 returns a MDC prediction, the prediction cycle controller 300 can put everything together as grouped items, using the MDC prediction to look up the model DB 340 for what to show for the MDC prediction. In some embodiments, the prediction cycle controller 300 can perform post-processing to group documents together, group lab results together, etc. and store them in the UM DB 350 for review by UM nurses.

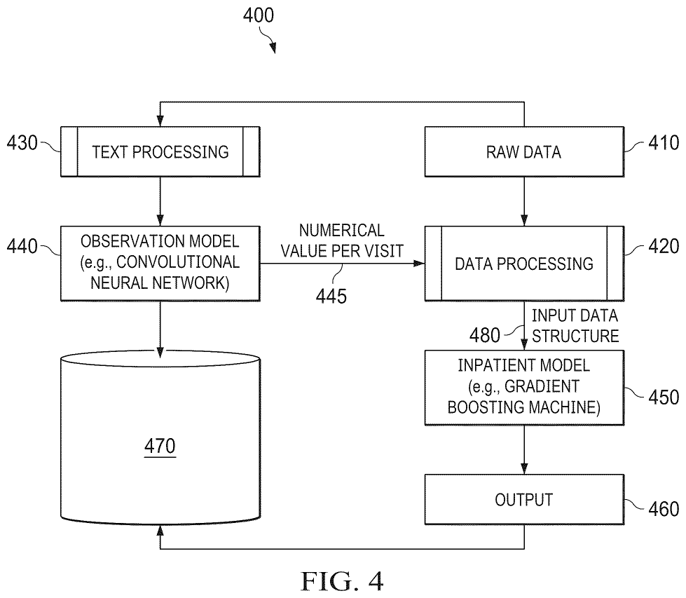

FIG. 4 is a diagrammatic representation of an admit status prediction process 400 configured for processing raw data 410 through an observation model 440 and/or an inpatient model 450 so as to generate an admit status prediction 460. In some embodiments, a data processing engine is configured for performing the process 400 on a computer (e.g., a server machine).

An example of a data source for the raw data 410 can be a document (e.g., a physician's note) that describes a patient's visit to a healthcare facility (e.g., a hospital, a clinic, etc.). Text data extracted from the raw data 410 undergoes text processing 430 performed by the observation model 440.

As alluded to above, an objective of this disclosure is to accurately predict a patient's hospital status, focusing specifically on the transitions between Inpatient and Observation/Outpatient statuses. Recognizing that the reasons for an Observation/Outpatient patient transitioning to Inpatient are typically different from an Inpatient moving to Observation/Outpatient, two distinct models, which are referred to as the observation model (e.g., the observation model 440) and the inpatient model (e.g., the inpatient model 450), are utilized to capture these differences more effectively.

As discussed below, the target labels for these models are binary: for the observation model, patients transitioning to ‘inpatient’ are labeled as such, while all other status changes or non-changes are labeled as ‘other’. Conversely, in the inpatient model, the labels are ‘observation’ and ‘current’, with ‘current’ covering all other scenarios. That is, these are binary classification models which only predict whether the status should transition to ‘inpatient’ or ‘observation’ respectively. Here, the output is how confident the model is that the patient should be in the respective status it's predicting on. If it falls below that confidence, the prediction is recorded as not transitioning to the status being predicted on (i.e., keeping the same status that they currently have). This distinction is important because it highlights the capability for the prediction architecture disclosed herein to easily expand to accommodate additional admission statuses, in an almost “plug-and-play” manner.

In some embodiments, the observation model 440 uses a convolutional neural network (CNN) to analyze medical documents for predicting status changes. A CNN is a network architecture for deep learning that learns directly from data. Generally, CNNs are useful for finding patterns in images to recognize objects, classes, and categories. In this case, however, the CNN can be particularly trained for performing the text processing 430 of the raw data 410.

In some embodiments, the text data thus extracted is kept in order and cleaned in a cleansing process to remove stop words (i.e., words that are used frequently and that have little meaning to a medical condition of interest). This cleansing process produces clean text data.

In some embodiments, using a subset of cases that have a particular medical condition of interest, the clean text data is run through a filtering process that utilizes a keyword extraction tool (e.g., KeyBERT, which is a Large Language Model (LLM) based tool for keyword extraction). The keyword extraction tool leverages BERT embeddings and basic cosine similarity to determine a set of words (e.g., keyword phrases) that have the highest correlation to the positive case. KeyBERT refers to a minimal method for keyword extraction with BERT. The keyword extraction is done by finding sub-phrases in a document that are the most similar to the document itself. First, document embeddings are extracted with BERT to get a document-level representation. Then, word embeddings are extracted for N-gram words/phrases. Finally, cosine similarity can be used to find the words/phrases that are the most similar to the document. The most similar words could then be identified as the words that best describe the entire document.

Other transformer models can also be used. For instance, for a given medical condition, Acute Blood Loss Anemia, a transformer model can examine data and determine what beneficial related words are associated with this medical condition and extract them from patient records. In one embodiment, the patient records are from a single healthcare facility (e.g., a hospital) with a sample size of at least 250 patients or more. Further, a single transformer model can be configured for identifying words associated with multiple medical conditions. In some embodiments, there can be multiple transformer models, each configured for identifying words associated with a particular medical condition.

Next, a defined set of fully documented phrases are removed, through another filtering process, from the list of words/phrases, resulting in a filtered list of words/phrases. These fully documented phrases (“FDP's”), which can be obvious medical terms used by physicians, provide the documentation for the patient's medical condition and need to be removed, so that the machine learning model can find only the evidence for the medical condition, rather than looking at the FDP's to make a prediction.

The filtering process cleans the text again to remove even more unnecessary words/phrases and results in a list of words/phrases that should only be considered in the machine learning model (e.g., keywords for a particular medical condition, for instance, “impairments,” “pediatricians,” “postnatal,” “prediabetic,” “shoulder,” “complaint,” “concern,” “plantar,” “aphasia,” etc.). Then, the filtered list of keywords/phrases is processed once again, through yet another filtering process, so that these keywords/phrases are arranged in their order. This ordering process allows a very directed set of keywords/phrases to be used in the machine learning model. This directed set allows the machine learning model to run faster from a smaller set of data.

Finally, the directed/ordered set of keywords/phrases is tokenized and padded, through a tokenization process, to create a final data set. The tokenization process may use any suitable tokenizer.

Table 1 below shows sample input data for a CNN Model.

TABLE 1

| | 0 | 1 | 2 | . . . |
|---|---|---|---|---|
| 0 | 0 | 0 | 0 | . . . |
| 1 | 0 | 0 | 0 | . . . |
| 2 | 0 | 0 | 0 | . . . |
| 3 | 0 | 0 | 0 | . . . |
| 4 | 0 | 0 | 0 | . . . |
| 5 | 85 | 425 | 95 | . . . |
| 6 | 0 | 0 | 0 | . . . |
| 7 | 0 | 0 | 0 | . . . |
| . . . | . . . | . . . | . . . | . . . |

In Table 1, the entries from left to right follow the order of words from a sentence in the input document. As discussed above, a non-limiting example of the document can be a physician note by a document who wrote a sentence that describes a patient (e.g., “A patient came in with a high fever . . . ”). The entries, from top to bottom, follow the order of the sentences in the document. Through the cleansing process discussed above, stop words not related to the medical condition of interest are removed. Through the first filtering process discussed above, statistically relevant words and phrases are found. Through the second filtering process discussed above, words that can serve as evidence of the medical condition are found (instead of words/phrases that document the medical condition of interest). Further, through the filtering process discussed above, only words/phrases that are directly related to the medical condition are kept. These pre-processing steps allow a very efficient tokenization. For instance, suppose that the word “large” can be transformed into a numerical value of 85, the word “blood” can be transformed into a numerical value of 425, and the word “loss” can be transformed into a numerical value of 95, and further suppose that the fifth entry for the list of ordered words contains three words “large blood loss,” then the fifth entry can be tokenized into 85, 425, 95, respectively, as shown in Table 1 above. Since other entries at the same first, second, and third positions do not have words, they are padded with zeros.

This final dataset is used to train a binary classification model for text. Following the above example, the final dataset is used as input to train a CNN model. The CNN model, in turn, produces a result for the patient associated with the document. Since the document describes the patient's visit, the output from the CNN model can be a numerical value 445 that indicates a likelihood that the patient's visit to the healthcare facility is related to the medical condition of interest. The numerical value 445 can be a number between 0 and 1. The CNN model thus trained can then be stored in a data store 470 (e.g., with the final output 460).

In some embodiments, a set of features particular to a certain medical condition of interest can be identified from the raw data 410 via a data processing operation 420. Examples of these features can include, but are not limited to, lab results, medications, tests, cultures, etc. Then, features attributes can be configured around each feature thus identified.

For instance, if hemoglobin is considered as a useful lab result, then hemoglobin-related feature attributes are determined, for instance, “minimum value,” “maximum value,” “number of times that the max value was above a given reference range,” and a number of other calculations. Such feature attributes can derive from medications, services, observations, etc., and processed to prepare the feature data as input to the inpatient model 450.

In some embodiments, a data processing engine running the data processing operation 420 may utilize historical data stored in tables (e.g., a table for medications, a table services, a table observations, etc.). The data processing engine can take these tables as input and processes them based on a configuration file which stores a list of what to process—what medications, what services, what observations, etc. The configuration file is specific to a particular medical condition. The data processing engine processes the tables to extract features of interest from the tables (e.g., medications, services, observations, etc.) based on the configuration file. The configuration file specifies what it needs (e.g., certain medical-condition-specific parameters/variables that need values) and the data processing engine calculates and uses the calculated values to determine a probability that the medical condition exists—i.e., a medical-condition-specific score, between 0 and 1, that the medical condition happened to the respective patient—each patient gets a score). The predictions can be stored in a new “results” table.

The CNN's output 445 (i.e., a numerical value per a patient's visit generated by the observation model 440) is considered an input feature and combined, through the data processing operation 420, with other feature values (e.g., information on labs, services, and medication that are being used on a patient) in a data structure 480. Table 2 below is an example of an input data structure 480.

TABLE 2
XGBoost Model Sample Input Data

| visitId | result | docOutput | bloodpressuresystolic_initial | . . . |
|---|---|---|---|---|
| 8904730_hosp1 | 0 | 0.000796 | 177 | . . . |
| 271711_hosp2 | 0 | 0.004077 | 111 | . . . |
| 5987424_hosp3 | 0 | 0.001668 | 110 | . . . |
| 7377557_hosp4 | 0 | 0.041904 | 157 | . . . |
| . . . | 0 | 0.001798 | 134 | . . . |

As shown in the example of Table 2, the “docOutput” column is populated (e.g., through the data processing operation 420) with the numerical value per visit output 445 from the observation model 440.

In some embodiments, the inpatient model 450 runs an XGBoost algorithm and takes the data structure 480 from the data processing operation 420 as input. More specifically, the inpatient model 450 is operable to process the input data structure 480 and determine, for each visit, whether the patient's visit concerns a particular medical condition of interest. The results, each of which is a numerical value between 0 and 1, are used to update the input data structure 480, as shown in Table 2.

As those skilled in the art can appreciate, predictions can be computationally expensive to make. Using a combination of a transformer model for keyword extraction and a tokenizer for transforming text values into numerical values, among various filtering processes, help to produce a streamlined input array to the CNN model discussed above. This array allows the CNN model to run very efficiently (e.g., reducing 15,000 columns of data into 3000 columns of data).

Further, because the input array contains data highly relevant to a medical condition of interest, the CNN model can perform better by producing more accurate predictions. Likewise, through the data processing operation 420, features in many different types of inputs (e.g., lab results, medications, tests, cultures, etc.) can be identified as being associated with the same patient and particular to a certain medical condition of interest. Combined with the per-visit predictions from the CNN model, the XGBoost model can generate a patient-specific score indicating a probability of the patient having the particular medical condition. The final output 460, a prediction on whether a medical condition of interest exists for a patient, can then be stored in the data store 470.

In some embodiments, the machine learning models disclosed herein are built on data points sourced from internal client databases. These data points are derived from EMRs, capturing when patients enter and exit specific statuses (i.e., when each patient transitions into and out of observation or inpatient statuses). The machine learning models disclosed herein can examine data points from many perspectives and look for evidence to validate what medical condition is documented (e.g., via the CDI technology described in the above-referenced U.S. Pat. No. 10,886,013, entitled “SYSTEMS AND METHODS FOR DETECTING DOCUMENTATION DROP-OFFS IN CLINICAL,” which is incorporated by reference herein).

As those skilled in the art can appreciate, the machine learning models disclosed herein can be implemented in various ways. As another non-limiting example, the CNN's first layer may employ a Gensim Word2Vec model for embedding generation. This model can be trained on roughly 800,000 text documents (e.g., outputs from the text processing operation 430 on the raw data 410) associated with Inpatient visits, encompassing several document types related to a visit. For data preprocessing, all dates and non-alphanumeric characters were removed using regex. Next, SpaCy was used to lemmatize the documents, reducing words down to their roots. This preprocessed data then served as the training set for the Word2Vec model. The CNN can ingest text documents processed in the same manner as the Word2Vec model's input. All of these documents can be concatenated to represent a single data point, ensuring each data point encompasses all documents from entry into observation until the exit.

In some embodiments, the Keras library can be utilized for constructing the CNN. The Keras-tuner package, especially its distributed hyperband tuning feature, was used to optimize layer configurations. The initial layer utilizes the pre-trained Word2Vec model to transform document texts into embeddings. These embeddings are sequentially passed through two convolutional layers, beach with 128 filters and a kernel size of 5. The activation function chosen for these layers is the rectified linear unit (ReLu). To prevent overfitting of the model, a one dimensional spatial dropout, with a 0.5 dropout rate, is applied after each convolutional layer, selectively dropping entire one dimensional feature maps.

After the convolutional and dropout layers, the architecture incorporates a one dimensional global max pooling layer. This layer reduces the dimensions by extracting the most important features. The outputs of this layer are then processed through a dense layer with 20 units, accompanied by an Exponential Linear Unit (ELU) activation function. Next is an additional dropout layer with a 0.35 dropout rate, aiding in the prevention of overfitting. The final layer is a dense unit with a sigmoid activation function, facilitating the binary classification of ‘Inpatient’ or ‘Other’ for patients that are currently in observation.

As another example, both the inpatient model and the observation model may use the Gradient Boosting Machine (GBM) in their model architecture. However, the inpatient model may include an extra feature, which is the output of the CNN. Besides this one feature, the inpatient and observation models may use all of the same features. As a non-limiting example, features used in the GBM model can include, but are not limited to, “document prediction,” “heart rate,” “age,” “bmi,” “temperature,” “blood pressure systolic,” “weight,” “sodium,” “alkaline Phosphate,” “drug,” “hct,” “protein,” “labs,” “magnesium,” “imaging,” “blood pressure diastolic,” “wound note,” “cbc panel,” “nurse note,” “mrsa culture,” etc. There can be more than a hundred features per a model and each subsequent model will have different features yet still solving the same problem.

In this example, the GBM is built using the XGBoost classifier model and it is hypertuned using RayTune. This was used to search for the optimal parameters over a predefined search space. As a non-limiting example, the search space for the RayTune job is as follows. The search space will change upon each build of the model.
Predictor: Specified to use the gpu for predictions, “gpu_predictor”
Objective: Set to “binary: logistic”, indicating a binary classification with logistic regression
Evaluation Metric: the metric “error” is used to compute the binary classification error
Tree Method: trees are built using the gpu accelerated histogram, “gpu_hist”
Number of classes: Set to 1, meaning the model predicts a binary output
Max Depth: Depth of the trees can be 3, 5, 7, or 8
Minimum Child Weight: This value can be 1, 2, 4, or 8
Subsample: The fraction of data which is sampled for tree building, which is between 0.9 and 1
Learning rate (eta): values between 1×10\hat{ }−7 and 0.4. This determines the step size in model updates.

In this example, RayTune outputs an optimized set of parameters given the example search space and input data. It would be extremely unlikely to produce the same parameters without the same input data. The following were the final optimized parameters for the observation model:
Predictor: Specified to use the gpu for predictions, “gpu_predictor”
Objective: Set to “binary: logistic”, indicating a binary classification with logistic regression
Evaluation Metric: the metric “error” is used to compute the binary classification error
Tree Method: trees are built using the gpu accelerated histogram, “gpu_hist”
Number of classes: Set to 1, meaning the model predicts a binary output
Max Depth: depth of the trees is set to a max of 8
Minimum Child Weight: This was set to 1
Subsample: The fraction of data which is sampled for tree building, which was set to 0.9995
Learning rate (eta): Set to a value of 0.08888. This determines the step size in model updates.

The GBM model undergoes a plurality of boosting rounds (e.g., 150), and the results from the evaluation set are utilized to compute the root mean square error (RMSE).

The inpatient model may undergo a quiet period (e.g., 16 hours) to wait for data to assimilate. The observation model does not have a quiet period.

The patient status prediction and the MDC prediction thus generated, along with explanations of how the predictions were made, can be presented as a visit summary through a user interface, stored in the UM database, and/or used to generate a clinical summary, as described below.

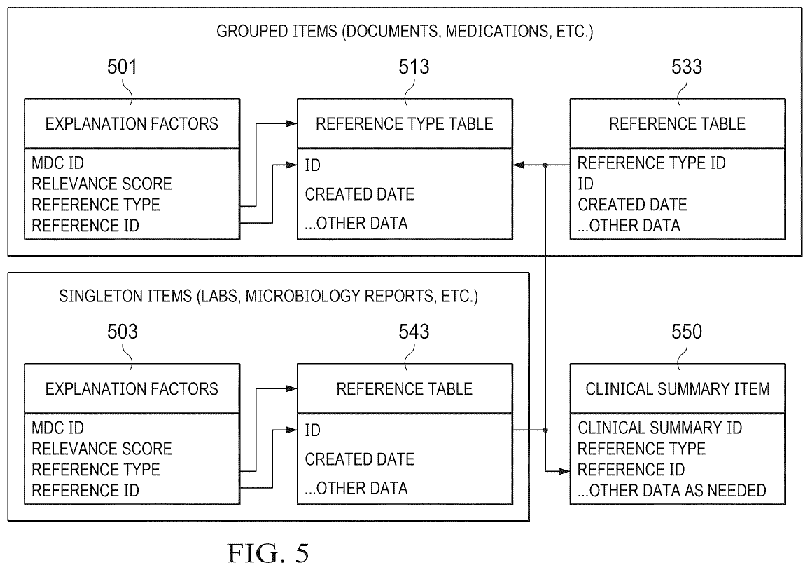

FIG. 5 is an example of what summary items may be grouped together (e.g., documents, medications, etc.) and what summary items may be presented as singleton items. In this example, both types of summary items (i.e., grouped items and singleton items) are keyed to the same MDC identifier (ID).

In some embodiments, there can be four types of summary items: keywords and phrases, observations, vitals, and services. Keywords and phrases can be associated with medical concepts, identified using concept unique identifiers (CUIs). Observations can include various types of labs, tests, procedures, medications, etc., for instance, lab ordered, procedures performed, tests conducted, white blood cell counts, etc. Vitals are those taken of a particular patient and may be timestamped. Services can include those ordered/provided for the patient (e.g., a physician's order for the MRI service, brain scan, etc.) that could be relevant to a MDC.

As those skilled in the art can appreciate, currently, patient cases are classified into Medicare Severity Diagnosis Related Groups (MS-DRGs). MDC is a grouping of similar MS-DRGs. Each of the MS-DRGs is defined by a particular set of patient attributes which include the principal diagnosis, any specific secondary diagnoses, procedures, sex and discharge status. Thus, each MDC is associated with a group of similar MS-DRGs, each of which defined by a set of patient attributes. In this disclosure, these MDC-specific attributes are referred to as explanation factors.

Referring back to FIG. 3, the model database 340 may store explanation factors for each possible MDC (referred to as explanatory clinical data). At runtime, the prediction cycle controller 330 may extract features (clinical data) from the model database 340, provide the extracted features to the MDCP 330, and receive a predicted MDC in response. The explanation factors for the predicted MDC can then be used to query the model database 340 for explanatory clinical data (e.g., medication orders, keywords and phrases, etc.). The explanation factors can be stored in the UM database 350 as metadata. In such cases, pointers to the explanatory clinical data (contained in the model DB 340) are stored in the UM database 350.

As illustrated in FIG. 5, the explanation factors can be stored in a table (e.g., tables 501, 503) that is linked to reference type tables (e.g., table 513), each for a particular type of explanation factors (e.g., CUIs, observations, vitals, and services). Each reference type table, in turn, is linked to a reference table (e.g., tables 533, 543) that stores details about each explanation factor (e.g., what medication is prescribed, when a vital sign is taken, what service is performed, etc.). As described below, these tables can be utilized by a control logic (e.g., a clinical summary resolver) to create a clinical summary (e.g., clinical summary 550).

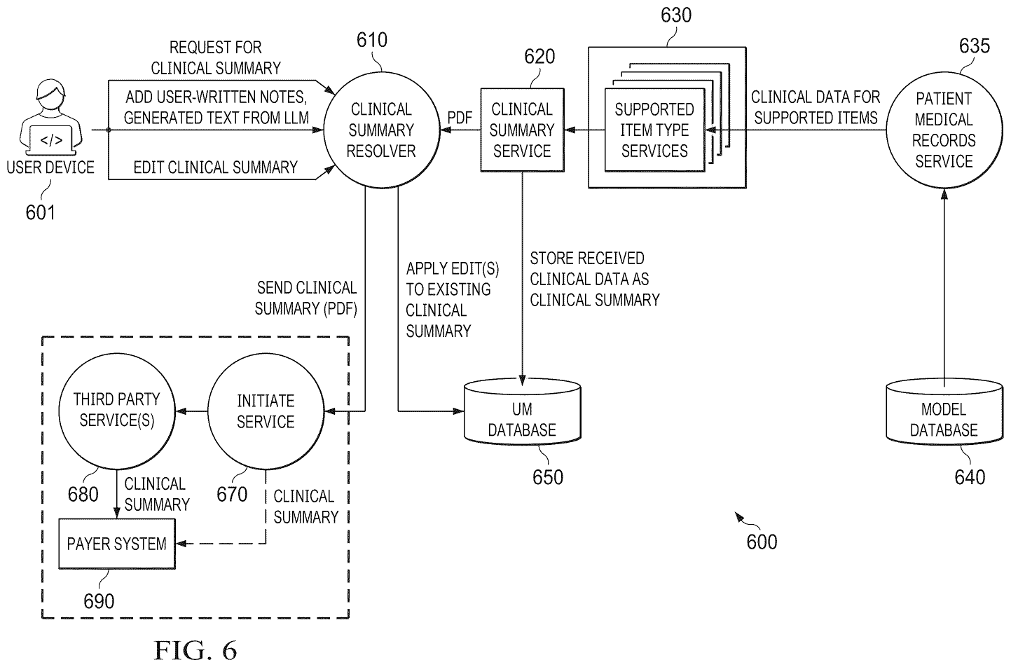

FIG. 6 illustrates an example of a system 600 for generating a clinical summary according to some embodiments disclosed herein. In this example, a UM nurse requests a clinical summary via a user interface on a user device 601. The user interface may implement a web portal or a client application of the system 600.

As a non-limiting example, the request for the clinical summary may be received by a clinical summary resolver 610 of the system 600. The clinical summary resolver 610, in turn, may call a clinical summary service 620 to generate a document (e.g., in the Portable Document Format (PDF)).

In response, the clinical summary service 329 calls a supported item type service(s) 630 to retrieve clinical data from the model database. There can be multiple supported item type services, each specific to a type of item (e.g., documentation, medication, labs, etc.). The supported item type service, in turn, calls a patient medical records service 635 to retrieve the clinical data from a model database 640.

In some embodiments, the clinical data can include previously generated predictions for the patient (i.e., the patient's current admit status prediction and the patient's MDC prediction) as well as from the actual clinical data for the patient (e.g., specific medication orders and administrations, specific labs and their results, the text of specific documents, etc.). As a non-limiting example, the patient-specific clinical data thus retrieved is stored in the UM database 650 as the patient's current clinical summary. The UM 650 database stores the metadata that allows for the exact generation of the PDF again, while the only concrete copy of that exact clinical data is stored in the model DB 640 and retrieved through the patient medical records service 635.

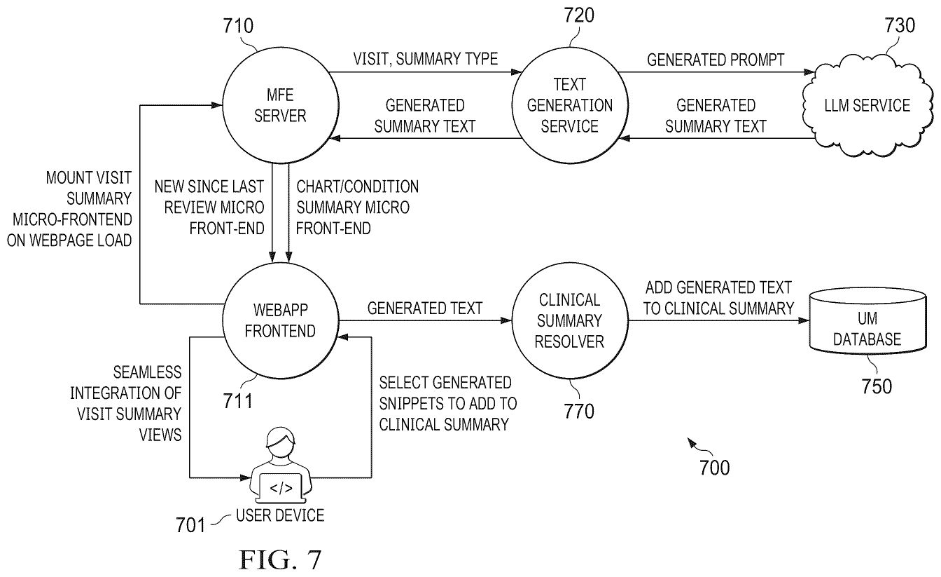

In some embodiments, the patient's current clinical summary can be edited through a web-based frontend user interface, which is referred to as a web application in FIG. 7. In one embodiment, the MFE server may include the system 100. In one embodiment, the flow shown in FIG. 7 does not involve the prediction cycle controller.

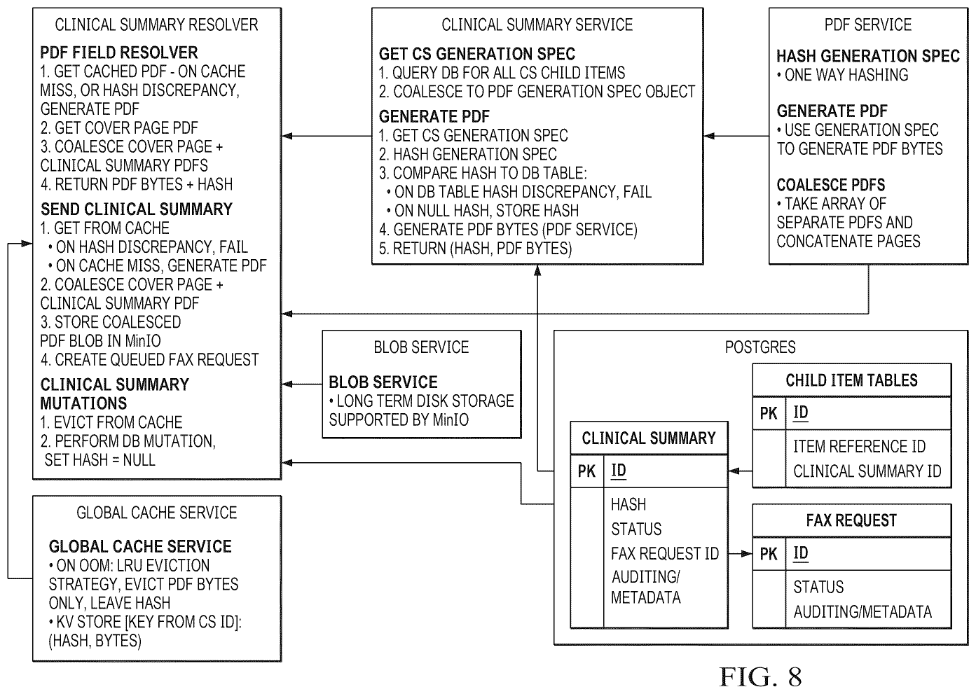

As shown in FIG. 7, within a web application 711, such a request may cause a micro-frontend program (e.g., the MFE server 710), while loading a web page on a user (e.g., an UM nurse) device 701, to request the system 700 to call a text generation service 720 with data about a patient's visit and the patient's visit summary. The text generation service 720, in turn, may call an LLM service 730 (e.g., ChatGPT or any suitable LLM services) to obtain summary text reflective of the patient's visit and the patient's visit summary. An example of a data model used by the clinical summary resolver in operation is shown in FIG. 8.

The generated summary text, which can include a chart/condition summary, is returned by the LLM service 730 to the text generation service 720 then to the MFE server 710. The system 700 is operable (e.g., through another micro-frontend program) to check what is new in the patient's chart/condition summary since the last time the patient was reviewed and the clinical summary was generated for the patient and present the new information as snippets on the web page.

In some embodiments, the UM nurse can select and add any of the generated snippets to the clinical summary for the patient. The UM nurse's selection can be communicated, via the web application 711, from the user device 701 to a clinical summary resolver 770 which stores the selected snippets as part of the clinical summary in the UM DB 750.

In some embodiments, in addition to allowing the UM nurse to edit the clinical summary, user-written notes and other documentations could also be added to the clinical summary. Once all the edits are resolved by the clinical summary resolver 770, the clinical summary (in a document form) can be communicated to a downstream computing facility (e.g., an initiate service 670) and/or an external computing facility. For instance, as shown in FIG. 6, a clinical summary can be communicated to a payer system 690 through various network communications means such as sending a fax using an electronic fax service, making an API call (e.g., via a REST API), and/or streaming through a data feed (e.g., an HL7 stream). A system implementing an embodiment of a method disclosed herein can be communicatively connected to the payer system 690 through one or more third-party services 680, as shown in FIG. 6.

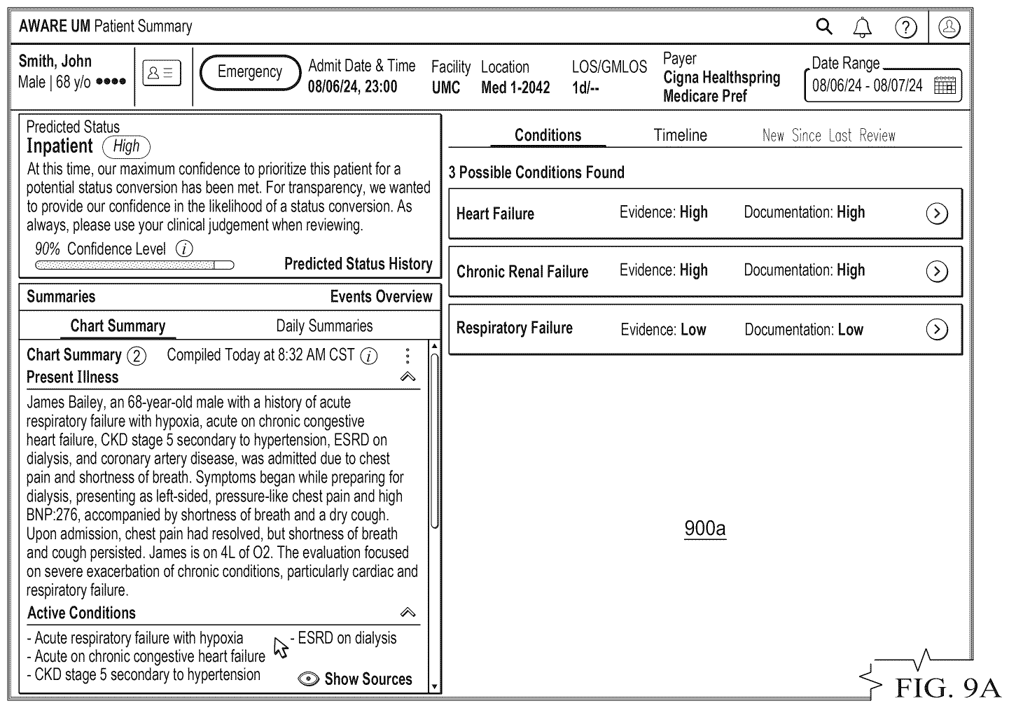

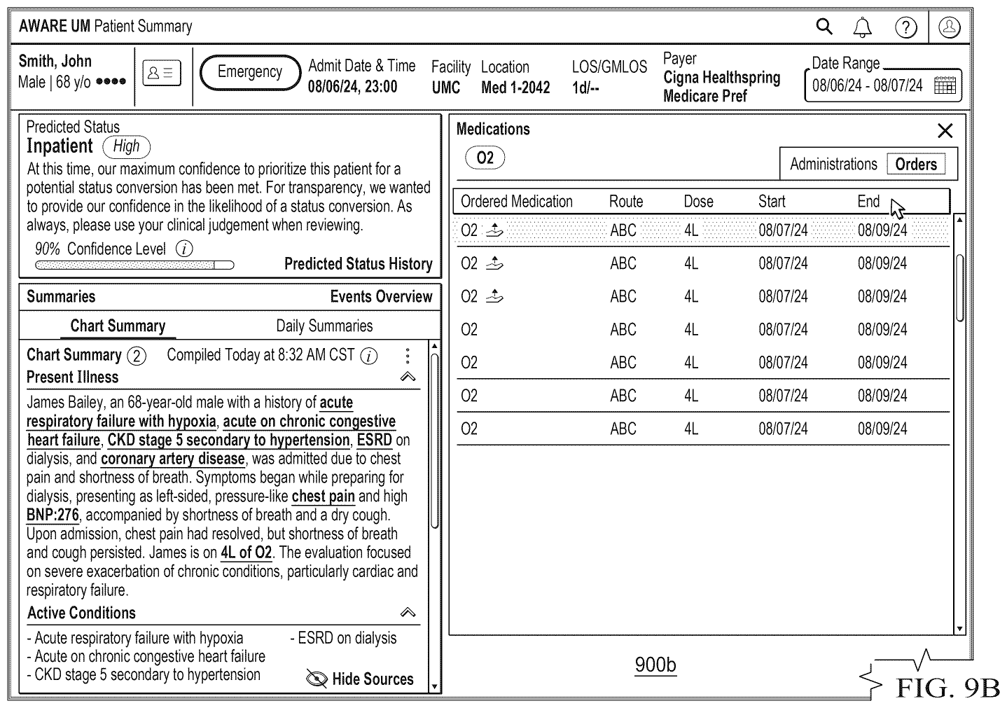

FIGS. 9A-B show example views of a user interface displaying a machine-generated clinical summary according to some embodiments disclosed herein. FIG. 9A shows an example view 900a of a machine-generated summary text returned by an LLM service (e.g., to the text generation service 720 then to the MFE server 710). FIG. 9B shows an example view 900b updated to show new information as snippets. In some embodiments, this can be done by leveraging Retrieval-Augmented Generation (RAG).

As those skilled in the machine learning art can appreciate, in RAG, embeddings can be used to retrieve relevant documents or pieces of information from a large corpus based on a user's query. In some embodiments, the system may examine patient medical records to identify those containing medical terms of interest or topic-related words (e.g., a topic relating to an MDC). Then, the system can instruct the RAG module to search patient medical records and find relevant data points. The data points thus obtained can be ordered by their respective relevancy. The system may select the most relevant ones (e.g., the first 20) of the data points as prompts and send the selected prompts to the LLM service. These prompts are instructions or inputs for an LLM to guide the LLM in generating a specific response, such as the text for a visit summary. These prompts act as a starting point, guiding the LLM to understand the task, tone, and context needed to produce the desired output, which is then returned by the LLM service to the system, for instance, for generating a view on the user interface to display the visit summary.

In some embodiments, the UM nurse can edit a machine-generated visit summary for a patient by, for instance, selecting and adding any of the generated snippets to the visit summary for the patient. The UM nurse's selection can be communicated, via the web application 711, from the user device 701 to a clinical summary resolver 770 which stores the selected snippets as part of the visit summary in the UM DB 750. These snippets can be linked or otherwise associated with the visit summary by mapping to the clinical summary's identifier.

In some embodiments, to enable a user to edit a machine-generated visit summary for a patient, the system is operable to tag the snippets (e.g., via HTTL tags in the source code of a web page on which the machine-generated visit summary for the patient is displayed) and, for each snippet thus tagged as an element of the web page, associate the element with a unique identifier. In this way, when the machine-generated visit summary is presented to a user via a user interface (e.g., the view 900b), the tagged elements are user-selectable.

When a tagged element is selected, the user interface is operable to display a corresponding window or frame that allows the user to view and/or edit details of the tagged element. For distribution, the system can generate a clinical summary in a distributable document format based on the visit summary thus generated (and/or user-edited). By deep-linking a machine-generated user-editable visit summary to other pieces of highly relevant information, the invention can improve a computer's functioning, both in terms of speed and quality (e.g., relevancy) in generating a visit summary for a patient in a concise, consistent, reliable, and reproducible way.

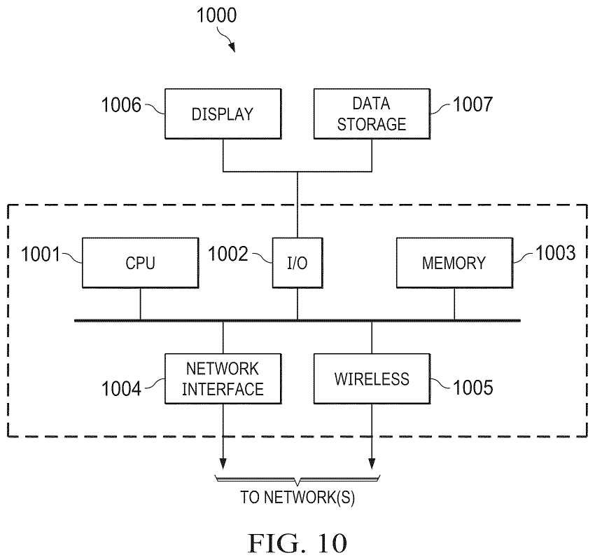

FIG. 10 depicts a diagrammatic representation of a data processing system for implementing an embodiment disclosed herein. As shown in FIG. 10, a data processing system 1000 may include one or more central processing units (CPU) or processors 1001 coupled to one or more user input/output (I/O) devices 1002 and memory devices 1003. Examples of I/O devices 1002 may include, but are not limited to, keyboards, displays, monitors, touch screens, printers, electronic pointing devices (for example, mouse, trackball, stylus, touch pad, etc.), or the like.

Embodiments discussed herein can be implemented in a computer communicatively coupled to a network (for example, the Internet), another computer, or in a standalone computer. As is known to those skilled in the art, a suitable computer can include a central processing unit (“CPU”), at least one read-only memory (“ROM”), at least one random access memory (“RAM”), at least one hard drive (“HD”), and one or more input/output (“I/O”) device(s). The I/O devices can include a keyboard, monitor, printer, electronic pointing device.

Examples of memory devices 1003 may include, but are not limited to, hard drives (HDs), magnetic disk drives, optical disk drives, magnetic cassettes, tape drives, flash memory cards, random access memories (RAMs), read-only memories (ROMs), smart cards, etc. The data processing system 1000 can be coupled to a display 1006, an information device 1007 and various peripheral devices (not shown), such as printers, plotters, speakers, etc. through I/O devices 1002. The data processing system 1000 may also be coupled to external computers or other devices through network interface 1004, wireless transceiver 1005, or other means that is coupled to a network such as a local area network (LAN), wide area network (WAN), or the Internet.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, feature or function). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary.

While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

ROM, RAM, and HD are computer memories for storing computer-executable instructions executable by the CPU or capable of being compiled or interpreted to be executable by the CPU. Suitable computer-executable instructions may reside on a computer-readable medium (e.g., ROM, RAM, and/or HD), hardware circuitry or the like, or any combination thereof. Within this disclosure, the term “computer-readable medium” is not limited to ROM, RAM, and HD and can include any type of data storage medium that can be read by a processor. For example, a computer-readable medium may refer to a data cartridge, a data backup magnetic tape, a floppy diskette, a flash memory drive, an optical data storage drive, a CD-ROM, ROM, RAM, HD, or the like. The processes described herein may be implemented in suitable computer-executable instructions that may reside on a computer-readable medium (for example, a disk, CD-ROM, a memory, etc.). Alternatively, the computer-executable instructions may be stored as software code components on a direct access storage device array, magnetic tape, floppy diskette, optical storage device, or other appropriate computer-readable medium or storage device.

Any suitable programming language can be used to implement the routines, methods or programs of embodiments of the invention described herein, including C, C++, Java, JavaScript, HTML, or any other programming or scripting code, etc. Other software/hardware/network architectures may be used. For example, the functions of the disclosed embodiments may be implemented on one computer or shared/distributed among two or more computers in or across a network. Communications between computers implementing embodiments can be accomplished using any electronic, optical, radio frequency signals, or other suitable methods and tools of communication in compliance with known network protocols.

Different programming techniques can be employed such as procedural or object oriented. Any particular routine can execute on a single computer processing device or multiple computer processing devices, a single computer processor or multiple computer processors. Data may be stored in a single storage medium or distributed through multiple storage mediums, and may reside in a single database or multiple databases (or other data storage techniques). Although the steps, operations, or computations may be presented in a specific order, this order may be changed in different embodiments. In some embodiments, to the extent multiple steps are shown as sequential in this specification, some combination of such steps in alternative embodiments may be performed at the same time. The sequence of operations described herein can be interrupted, suspended, or otherwise controlled by another process, such as an operating system, kernel, etc. The routines can operate in an operating system environment or as stand-alone routines. Functions, routines, methods, steps, and operations described herein can be performed in hardware, software, firmware, or any combination thereof.

Embodiments described herein can be implemented in the form of control logic in software or hardware or a combination of both. The control logic may be stored in an information storage medium, such as a computer-readable medium, as a plurality of instructions adapted to direct an information processing device to perform a set of steps disclosed in the various embodiments. Based on the disclosure and teachings provided herein, a person of ordinary skill in the art will appreciate other ways and/or methods to implement the invention.

It is also within the spirit and scope of the invention to implement in software programming or code an of the steps, operations, methods, routines or portions thereof described herein, where such software programming or code can be stored in a computer-readable medium and can be operated on by a processor to permit a computer to perform any of the steps, operations, methods, routines or portions thereof described herein. The invention may be implemented by using software programming or code in one or more digital computers, by using application specific integrated circuits, programmable logic devices, field programmable gate arrays, optical, chemical, biological, quantum or nanoengineered systems, components and mechanisms may be used. In general, the functions of the invention can be achieved by any means as is known in the art. For example, distributed, or networked systems, components and circuits can be used. In another example, communication or transfer (or otherwise moving from one place to another) of data may be wired, wireless, or by any other means.

A “computer-readable medium” may be any medium that can contain, store, communicate, propagate, or transport the program for use by or in connection with the instruction execution system, apparatus, system or device. The computer-readable medium can be, by way of example only but not by limitation, an electronic, magnetic, optical, electromagnetic, infrared, or semiconductor system, apparatus, system, device, propagation medium, or computer memory. Such computer-readable medium shall be machine readable and include software programming or code that can be human readable (e.g., source code) or machine readable (e.g., object code). Examples of non-transitory computer-readable media can include random access memories, read-only memories, hard drives, data cartridges, magnetic tapes, floppy diskettes, flash memory drives, optical data storage devices, compact-disc read-only memories, and other appropriate computer memories and data storage devices. In an illustrative embodiment, some or all of the software components may reside on a single server computer or on any combination of separate server computers. As one skilled in the art can appreciate, a computer program product implementing an embodiment disclosed herein may comprise one or more non-transitory computer-readable media storing computer instructions translatable by one or more processors in a computing environment.

A “processor” includes any hardware system, mechanism, or component that processes data, signals or other information. A processor can include a system with a central processing unit, multiple processing units, dedicated circuitry for achieving functionality, or other systems. Processing need not be limited to a geographic location, or have temporal limitations. For example, a processor can perform its functions in “real-time,” “offline,” in a “batch mode,” etc. Portions of processing can be performed at different times and at different locations, by different (or the same) processing systems.

It will also be appreciated that one or more of the elements depicted in the drawings/figures can also be implemented in a more separated or integrated manner, or even removed or rendered as inoperable in certain cases, as is useful in accordance with a particular application. Additionally, any signal arrows in the drawings/figures should be considered only as exemplary, and not limiting, unless otherwise specifically noted.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited to only those elements but may include other elements not expressly listed or inherent to such process, product, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, including the claims that follow, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein and throughout the claims that follow, the meaning of “in” includes “in” and “on” unless the context clearly dictates otherwise. The scope of the present disclosure should be determined by the following claims and their legal equivalents.

What is claimed is:

1. A method, comprising:
performing, by a computer for each respective patient of a plurality of prediction-eligible patients of a healthcare provider:
determining, by a first machine learning model based on observations of the plurality of prediction-eligible patients, an admit status prediction (ASP) for the respective patient;
determining, by a second machine learning model utilizing the ASP and major diagnosis category (MDC) prediction features extracted from patient visits eligible for the ASP, a MDC prediction (MDCP) for the respective patient; and
extracting, from a database, clinical data for the respective patient;
mapping, by the computer utilizing the MDCP, individual clinical data points in the clinical data for the respective patient thus extracted into grouped items; and
generating, by the computer, a visit summary for the respective patient, the visit summary including the ASP, the MDCP, and an explanation of how the ASP and the MDCP were made based at least on the grouped items.

2. The method according to claim 1, further comprising:
generating, utilizing the visit summary, a clinical summary in a distributable document format.

3. The method according to claim 1, wherein the explanation comprises a plurality of explanation factors describing the MDC.

4. The method according to claim 1, wherein the visit summary further comprises a singleton item.

5. The method according to claim 1, wherein the first machine learning model comprises a multi-class classifier and wherein the second machine learning model comprises a binary classifier.

6. The method according to claim 1, wherein the observations comprise at least one of: a date when a current status was established, a date when the current status was changed, any change in severity of a patient's illness, or any change in the severity of the patient's symptoms.

7. The method according to claim 1, further comprising:
providing a user interface with a user interface element for editing the visit summary.

8. A system, comprising:
a processor;
a non-transitory computer-readable medium; and
instructions stored on the non-transitory computer-readable medium and translatable by the processor for performing:
for each respective patient of a plurality of prediction-eligible patients of a healthcare provider:
determining, by a first machine learning model based on observations of the plurality of prediction-eligible patients, an admit status prediction (ASP) for the respective patient;
determining, by a second machine learning model utilizing the ASP and major diagnosis category (MDC) prediction features extracted from patient visits eligible for the ASP, a MDC prediction (MDCP) for the respective patient; and
extracting, from a database, clinical data for the respective patient;
mapping, utilizing the MDCP, individual clinical data points in the clinical data for the respective patient thus extracted into grouped items; and
generating a visit summary for the respective patient, the visit summary including the ASP, the MDCP, and an explanation of how the ASP and the MDCP were made based at least on the grouped items.

9. The system of claim 8, wherein the instructions are further translatable by the processor for:
generating, utilizing the visit summary, a clinical summary in a distributable document format.

10. The system of claim 8, wherein the explanation comprises a plurality of explanation factors describing the MDC.

11. The system of claim 8, wherein the visit summary further comprises a singleton item.

12. The system of claim 8, wherein the first machine learning model comprises a multi-class classifier and wherein the second machine learning model comprises a binary classifier.

13. The system of claim 8, wherein the observations comprise at least one of: a date when a current status was established, a date when the current status was changed, any change in severity of a patient's illness, or any change in the severity of the patient's symptoms.

14. The system of claim 8, wherein the instructions are further translatable by the processor for:
providing a user interface with a user interface element for editing the visit summary.

15. A computer program product comprising a non-transitory computer-readable medium storing instructions translatable by a processor for performing:
for each respective patient of a plurality of prediction-eligible patients of a healthcare provider:
determining, by a first machine learning model based on observations of the plurality of prediction-eligible patients, an admit status prediction (ASP) for the respective patient;
determining, by a second machine learning model utilizing the ASP and major diagnosis category (MDC) prediction features extracted from patient visits eligible for the ASP, a MDC prediction (MDCP) for the respective patient; and
extracting, from a database, clinical data for the respective patient;
mapping, utilizing the MDCP, individual clinical data points in the clinical data for the respective patient thus extracted into grouped items; and
generating a visit summary for the respective patient, the visit summary including the ASP, the MDCP, and an explanation of how the ASP and the MDCP were made based at least on the grouped items.

16. The computer program product of claim 15, wherein the instructions are further translatable by the processor for:
generating, utilizing the visit summary, a clinical summary in a distributable document format.

17. The computer program product of claim 15, wherein the explanation comprises a plurality of explanation factors describing the MDC.

18. The computer program product of claim 15, wherein the first machine learning model comprises a multi-class classifier and wherein the second machine learning model comprises a binary classifier.

19. The computer program product of claim 15, wherein the observations comprise at least one of: a date when a current status was established, a date when the current status was changed, any change in severity of a patient's illness, or any change in the severity of the patient's symptoms.

20. The computer program product of claim 15, wherein the instructions are further translatable by the processor for:
providing a user interface with a user interface element for editing the visit summary.
